(19) United States
(12) Patent Application Publication
(10) Pub. No.: US 2022/0105334 A1
(43) Pub. Date: Apr. 7, 2022

(54) DEVICES, SYSTEMS AND METHODS FOR AN IMPLANTABLE DRUG DELIVERY DEVICE

(71) Applicant: Mott Corporation, Farmington, CT (US)

(72) Inventors: James K. Steele, Rockfall, CT (US); Vincent Palumbo, East Granby, CT (US); Sean Kane, Cranberry Township, PA (US); Aravind Mohanram, Avon, CT (US)

(21) Appl. No.: 17/418,984

(22) PCT Filed: Dec. 24, 2019
(86) PCT No.: PCT/US2019/068496

§ 371 Date: Jun. 28, 2021

Related U.S. Application Data
(60) Provisional application No. 62/785,973, filed on Dec. 28, 2018.

(51) Int. Cl. A61M 39/04; A61M 39/02; A61M 5/168; A61M 5/142; A61M 5/148

(52) U.S. Cl. CPC A61M 39/04; A61M 39/0247; A61M 5/16818; A61M 2039/027; A61M 5/148; A61M 2039/0261; A61M 5/14276

(57) ABSTRACT

The present disclosure relates generally to the field of medical devices and drug delivery. In particular, the present disclosure relates to implantable medical devices, systems and methods for controlled and consistent drug release through a porous body into a patient.

### FIELD

The present disclosure relates generally to the field of medical devices and drug delivery. In particular, the present disclosure relates to implantable medical devices, systems and methods for controlled and consistent drug release through a porous body into a patient.

### BACKGROUND

Many drugs used for treating chronic medical conditions may have delivery limitations. For example, such drugs may have a relatively large molecular weight or may be fragile such that the drug cannot be delivered orally, and therefore must be delivered by another route, such as via injection. Additionally, drugs that have relatively short half-lives or are administered in small doses that rapidly vacate the body such that they must be frequently re-administered to the patient.

Intravenous administration by a medical professional may be a safe and reliable method for administering drugs. In some cases, patients may frequently self-administer drugs by subcutaneous or intramuscular injection (e.g., several times a week). However, this type of therapy may be associated with problems such as pain at the site of injection, injection site reactions, infections, lack of compliance with dosing schedule, and lack of compliance with dosing amounts.

Drug delivery implant devices may provide sustained drug release without compliance concerns. Current implant devices may be associated with an inconsistent delivery of the drug over time by, e.g., an initial high dosage of the drug as the device is implanted, exposed to the body, and as the drug erodes. Such an initial high dosage after implantation may be similar to an injection schedule, which subjects the patient to highs and lows of drug exposure rather than a consistent sustained drug delivery. Some implant devices may use electronic and/or mechanical pumps or other delivery mechanisms that can increase the risk of infections and moving parts failure.

A variety of advantageous medical outcomes may be realized by the medical devices, systems, and methods of the present disclosure, which facilitate drug delivery to a patient. Although current technologies provide important advantages over traditional daily injections, a need currently exists for implantable devices that provide safe, consistent, and sustained drug delivery to a patient.

### SUMMARY

Embodiments of the present disclosure may assist generally with drug delivery without the need for a patient to be mindful of a drug administration schedule, painful injections, or inconsistent and unsustainable drug delivery devices. In one embodiment, an implantable drug delivery device may include a housing. A reservoir may be within the housing and may be configured to contain a fluid. A homogenous porous body having non-uniform pores of about 0.1 microns to about 100 microns may be at a first end of the housing and may be in fluid communication with the reservoir. A septum may be at a second end of the housing in fluid communication with the reservoir. The reservoir may extend into the porous body. The porous body may include a convex body. One or more filaments may be disposed on the drug delivery device that may be configured to attach the drug delivery device to a tissue. The housing may be curved to substantially match an anatomy of a patient. A channel may be disposed on the housing that may be configured to accept a suture that may be configured to anchor the implantable drug delivery device to a tissue. The porous body may include a material selected from the group consisting of stainless steel, glass, titanium, any biocompatible metal alloy, ceramic, and polymers. The porous body may include a selective laser sintered metal. The porous body may include an additive metal. The porous body may make up the housing.

In another aspect, an implantable drug delivery device may include a housing. A storage reservoir may be within the housing. A flexible membrane may be within the storage reservoir that may separate a fluid compartment configured to contain a fluid for delivery and a waste compartment configured to contain a waste. A first septum may be at a first end of the housing in fluid communication with the fluid compartment. A second septum may be at the first end of the housing in fluid communication with the waste compartment. A porous body may be at a second end of the housing. A fluid reservoir may be within the porous body in fluid communication with the storage reservoir. A fluid check valve may be in fluid communication with the fluid compartment and the fluid reservoir. The fluid check valve may be configured to allow flow substantially in a direction from the fluid compartment to the fluid reservoir. A waste check valve may be in fluid communication with the waste compartment and the fluid reservoir. The waste check valve may be configured to allow flow substantially in a direction from the fluid reservoir to the waste compartment. A lock may be configured to block fluid communication from the fluid compartment, through the fluid check valve, and into the fluid reservoir. A slidable member may be at an end of the porous body substantially opposing the storage reservoir and slidable within the fluid reservoir. The slidable member may be configured to be user-engageable to decrease a volume of the fluid reservoir. The slidable member may have a resting configuration where the slidable member may be substantially external to the fluid reservoir. The slidable member may have an engaged configuration where the slidable member may be substantially within the fluid reservoir. A lock may be configured to prevent the slidable member from sliding within the fluid reservoir.

In another aspect, an implantable drug delivery device may include an expandable member. A reservoir may be within the expandable member that may be configured to contain a fluid. A porous body may be at a first end of the expandable member in fluid communication with the reservoir. The expandable member may have an expanded configuration when a fluid is delivered into the reservoir. The expandable member may have a collapsed configuration when a fluid is removed from the reservoir. A septum may be disposed on the expandable member. A puncture-proof membrane may be disposed within the expandable member substantially opposing the septum. The porous body may be an annulus about the septum. A rigid housing may be about the expandable member.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a reservoir containing a fluid porous body, according to an embodiment of the present disclosure.

FIG. 2 illustrates a chart of the delivery rate of the fluid from the reservoir through the porous body of FIG. 1.

FIG. 3 illustrates an implantable drug delivery device with a reservoir within a porous body, according to an embodiment of the present disclosure.

FIG. 4 illustrates an implantable drug delivery device including a threaded filling port, according to an embodiment of the present disclosure.

FIG. 5 illustrates an implantable drug delivery device including a septum, according to an embodiment of the present disclosure.

FIG. 6 illustrates an implantable drug delivery device including a housing and a porous body at an end, according to an embodiment of the present disclosure.

FIG. 7A illustrates a curved implantable drug delivery device including a housing and a rounded porous body, according to an embodiment of the present disclosure.

FIG. 7B illustrates a syringe that may be used to deliver a fluid through a septum into the reservoir.

FIG. 7C depicts an expanded view of the septum tip;

FIG. 8A illustrates a use of the device of FIG. 7, where an incision is first made in the patient.

FIG. 8B shows the device of FIG. 7 into the muscle via sutures within the subcutaneous tissue layer.

FIG. 8C depicts a medical professional scanning the implantation site on the patient to ensure proper implantation of the device and/or to locate the device.

FIG. 8D depicts the medical professional loading a reservoir of the device with a fluid via a syringe percutaneously through the skin of the patient.

FIG. 8E depicts the medical professional making an incision in the patient to remove and/or replace the device in the patient.

FIG. 9A illustrates an implantable drug delivery device including a storage reservoir, according to an embodiment of the present disclosure.

FIG. 9B shows the storage reservoir filled with a fluid drug in the fluid compartment.

FIG. 9C shows the storage reservoir partially filled with a fluid drug in the fluid compartment and partially filled with was in the waste compartment.

FIG. 9D shows the waste compartment expanded to occupy the entire volume of the fluid reservoir, while the fluid drug compartment is reduced to its smallest possible size.

FIG. 9E depicts a first septum at a first end of the housing and in fluid communication with the fluid compartment.

FIG. 10 illustrates a use of the device of FIGS. 9A-9E.

FIG. 11A illustrates an implantable drug delivery device including an expandable member, according to an embodiment of the present disclosure.

FIG. 11B illustrates an implantable drug delivery device including an expandable member, according to an embodiment of the present disclosure.

FIG. 12 illustrates a chart of delivery rates of various fluids through porous bodies, according to embodiments of the present disclosure.

FIG. 13 illustrates a chart of delivery rates of various fluids through porous bodies, according to embodiments of the present disclosure.

FIG. 14A illustrates an implantable drug delivery device including a cap, according to an embodiment of the present disclosure.

FIG. 14B illustrates the implantable drug delivery device including a cap, according to an embodiment of the present disclosure.

FIG. 15 is a graph that depicts the results for MELOXICAM® delivery to male and female dogs using the device detailed herein.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to medical devices and systems for implantable drug delivery devices in the subcutaneous and muscle tissue, it should be appreciated that such medical devices and systems may be used in a variety of anatomies which require device implantation and drug delivery.

As used herein, the term “drug” includes fluids and other deliverable materials that contain a medically active component, and may also include other materials, e.g., a nutrient, a solution, or the like. The term “drug” may be used interchangeably herein with the term “fluid” throughout discussions of device structure and fluid mechanics.

As used herein, the terms “patient,” “user,” and “medical professional” may be interchangeable. Specifically, a user may also be a medical professional and a patient may also be a user.

As used herein, the term “patient” may refer to a human, a domesticated pet, livestock, a mammal, an untamed animal, a wild animal, or the like.

As used herein, the term “distal” refers to the end farthest away from the medical professional when introducing a device into a patient, while the term “proximal” refers to the end closest to the medical professional when introducing a device into a patient.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

A patient may enjoy a painless and task-free drug delivery after receiving an implanted drug delivery device. Such a device may deliver a consistent and sustained amount of drug into the patient by implanting the device containing a drug-filled reservoir that may be passively released through a porous body over time.

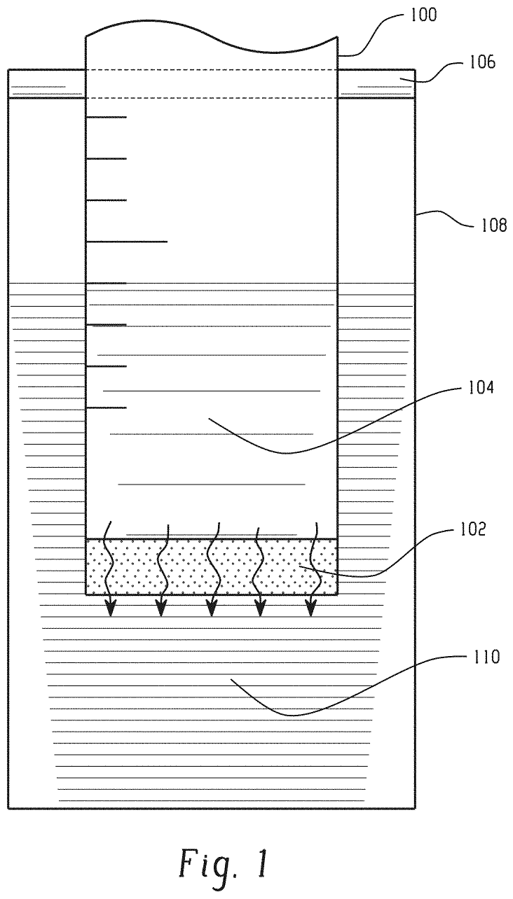

With reference to FIG. 1, a system embodiment for measuring and/or testing delivery of a drug according to the present disclosure is illustrated. The system includes a proximally closed syringe 100 having a porous body 102 at a distal end and containing a drug 104 (e.g., 0.5 mL of meloxicam having a concentration of about 10 mg/mL). The syringe 100 is inserted into a vial 108 through an o-ring 106. The drug 104 passively diffuses through the porous body 102 into a solution 110, which contains a phosphate buffer solution (e.g., 1.5 mL of the solution 110). The height of the fluid 104 within the syringe 100 substantially matches the height of the solution 110 within the vial 108 such that there is no substantial pressure differential head between the fluid 104 and the solution 110, allowing for passive diffusion to be the primary mode of material transport. Over time, e.g., daily, the syringe 100 is removed from the vial 108 and the concentration of the drug 104 in the solution 110 is measured, e.g., by using an ultraviolet spectrometer, a mass spectroscope, a liquid chromatography column, and/or the like. The syringe 100 is then inserted into a second vial (not shown) containing a new solution. These steps are repeated until the drug is consumed or the procedure is ceased. The procedure may be performed at e.g., room temperature, average human body temperature (about 37° C.), average canine body temperature (about 39° C.), or the like.

When various embodiments are in use, a fluid may diffuse through a porous body, for example, from a fluid reservoir, through the porous body, and into a patient. Generally, diffusion is the process of the random motion of molecules by which there is a net flow of matter from a region of higher concentration of a fluid to a region of lower concentration of a fluid. Diffusion may occur in two directions. For example, a first fluid (e.g., a drug) may diffuse from a fluid reservoir, through the porous body, and into a patient, while a second fluid (e.g., a waste or a solution) may diffuse from a patient, through the porous body, and into the fluid reservoir. The rate of flow of a diffusing fluid may be found to be proportional to a concentration gradient following the fixed law of diffusion.

In various embodiments, porous bodies may include porous open cell structures that are used for the controlled movement of fluids (i.e., diffusion of fluids and/or filtering of fluids). These structures may be formed using conventional techniques, such as by compacting metallic or ceramic powder or particles to form a pressed compact body and then sintering the body to form a coherent porous structure. Particle size, compaction force, sintering time, and sintering temperature may all influence pore size and mechanical properties. Generally, pore size is an important factor in the ability of a sintered structure to filter fluids and control the rate of fluid flow and/or diffusion through the sintered structure. In various embodiments, porous bodies may have interconnected pores of average size ranging from about 0.1 μm to about 100 μm. The overall size of these devices may vary, for example, from about 3 mm width or diameter by about 10 mm long for smaller subjects, to about 30 mm width or diameter by about 75 mm long or larger for larger subjects.

Characteristics of porous bodies of metal, ceramics, or other media are dependent on a number of factors, including the particular powder used, the green density (a ratio of powder volume to the external volume of the part), the sintering conditions employed, the configuration of the media, or the like. Depending on the application, important physical characteristics of the media may include its resistance to corrosion (e.g., from reaction with a wide range fluids), mechanical strength, and the ability to withstand various temperatures. Porous bodies may comprise materials such as stainless steel, titanium, a biocompatible alloy, an alloy, silica, glass, ceramics, polymers, polyether ether ketone, a combination thereof, or the like. Porous bodies of the present disclosure feature depth filtration between a reservoir and the external environment. This depth filtration is a tortuous path through a homogenous wall that has varying pore sizes. Average pore size of a porous body is controlled by the size of particles of the pre-sintered powder, temperature of the sintering, and sintering time. The porous bodies have a tortious fluid pathway to and from the reservoir, as opposed to many medical devices that may include a membrane with straight-through holes for filtration. An exemplary range of pore sizes for the porous bodies herein may be, for example, about 0.1 microns to about 5 microns, about 0.1 microns to about 20 microns, or about 0.1 microns to about 100 microns.

A single porous body may have a wide range of pore sizes, resulting in a porous body that is less susceptible to clogging than a porous body having pores of substantially the same size that could be clogged by a single fluid of substantially matching molecular size. Converse to a typical medical membrane, the porous bodies herein have a mean filtration value (i.e., an average pore size of the range of pore sizes of a porous body) rather than an absolute filtration value (i.e., all pores of a porous body being substantially the same size). A mean pore size for a porous body may vary with the viscosity and size of a compound within a fluid. For example, a porous body may include pores having a range of size of about 0.1 microns to about 2 microns or a range of size of about 0.1 microns to about 2 microns, about 0.1 microns to about 20 microns, about 0.1 microns to about 5 microns, or about 0.1 microns to about 100 microns.

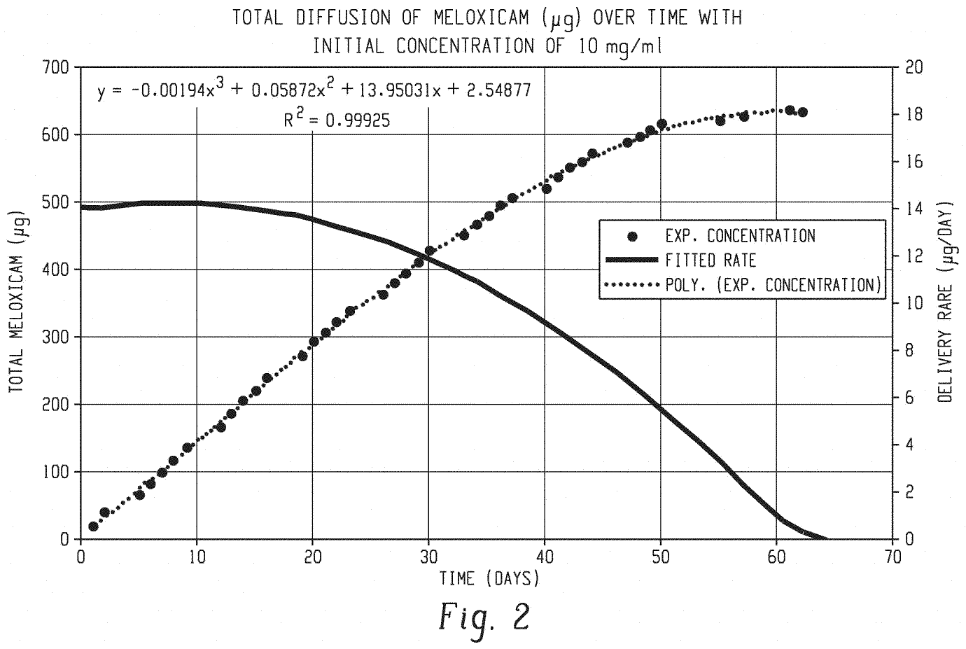

With reference to FIG. 2, a chart of fluid delivery rate from the syringe through the porous body of FIG. 1 is illustrated. The x-axis displays the amount of time (in a number of days) passed for each data point's measurement displayed on the dotted line having a positive slope. The solid cumulation concentration curve displays the slope of the dotted line of data points. The left y-axis displays a cumulative concentration of the fluid (meloxicam in micrograms (μg)) in the solution. The right y-axis displays a delivery rate of the fluid in μg/day (slope of the cumulation concentration curve), which is displayed by the solid line having a negative slope.

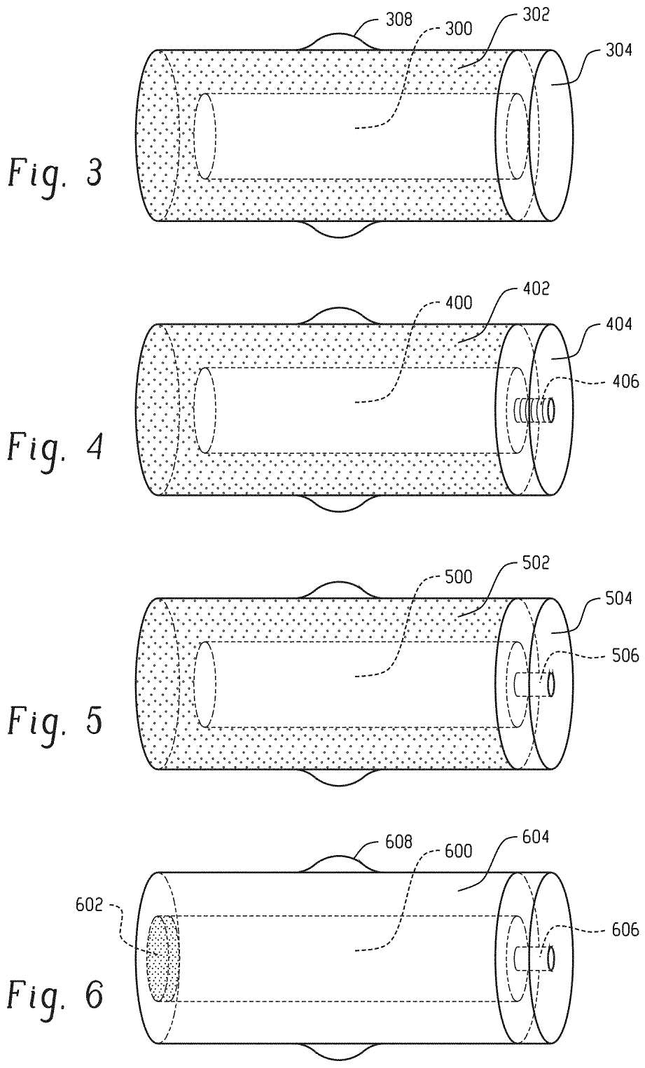

With reference to FIG. 3, an implantable drug delivery device according to an embodiment of the present disclosure is illustrated that includes a reservoir 300 within a porous body 302. The porous body 302 acts as a housing for the reservoir 300 and may generally contain a fluid within the reservoir 300. The porous body 302 is closed at an end by a solid endcap 304 that is welded or otherwise attached to the porous body 302. The porous body 302 is substantially cylindrical. The reservoir 300 is in fluid communication with the porous body 302, and the porous body 302 is in fluid communication with the external atmosphere (e.g., the body of a patient). Surgical loop filaments 308 are optionally attached to the porous body 302 for a medical professional to fix the device (e.g., tie, anchor, adhere, or the like) to an anatomy and/or deliver/remove the device to/from the implant site.

With reference to FIG. 4, an implantable drug delivery device according to an embodiment of the present disclosure is illustrated that includes a reservoir 400 within a porous body 402. The device of FIG. 4 is substantially similar to that of FIG. 3 and further includes a threaded fill port 406 in the endcap 404 for a fluid to be delivered into the reservoir 400.

With reference to FIG. 5, an implantable drug delivery device according to an embodiment of the present disclosure is illustrated that includes a reservoir 500 within a porous body 502. The device of FIG. 5 is substantially similar to that of FIGS. 3 and 4 but includes a septum 506 in the endcap 504 for passage by a syringe to deliver fluid into or from the reservoir 500.

With reference to FIG. 6, an implantable drug delivery device according to an embodiment of the present disclosure is illustrated that includes a reservoir 600 within a housing 604. The housing 604 is substantially cylindrical and contains a fluid within the reservoir 600. The housing 604 includes a porous body 602 at an end. The reservoir 600 is in fluid communication with the porous body 602, and the porous body 602 is in fluid communication with the external atmosphere (e.g., the body of a patient). In this embodiment, the surgical loop filaments 608 are attached to the housing 604.

In various embodiments, a fluid delivery rate may be altered by a number of factors. Such factors may include the molecular size of a fluid and/or solution, temperature, viscosity, and porous body structure such as pore size, wall thickness, and surface area. For example, to increase the diffusion rate of a fluid through a porous body, a larger external surface area and/or a lower porous body wall thickness may be selected.

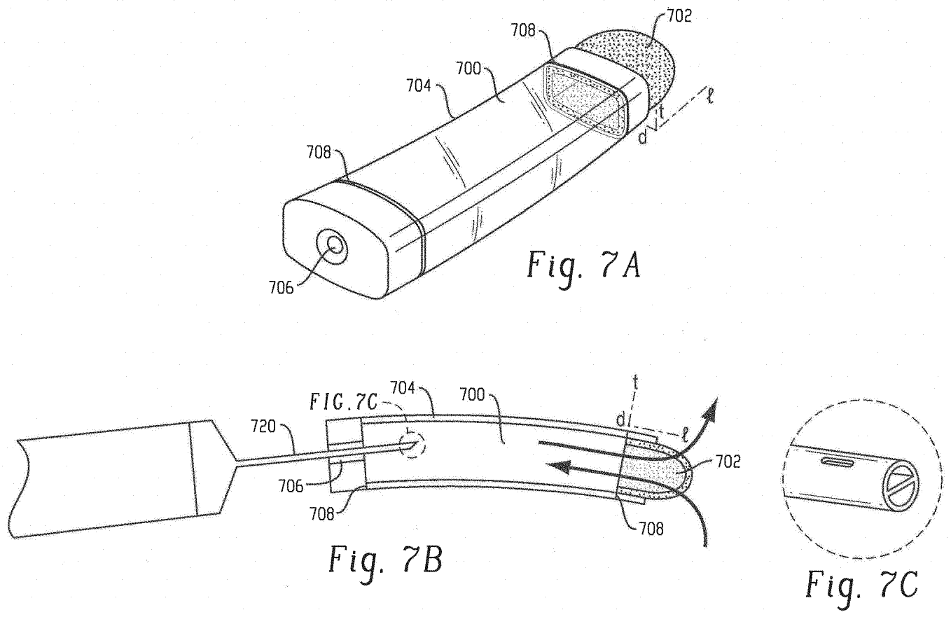

With reference to FIG. 7, an implantable drug delivery device according to an embodiment of the present disclosure is illustrated that includes a reservoir 700 within a housing 704 configured to contain a fluid. FIG. 7A illustrates a curved implantable drug delivery device including a housing and a rounded porous body, according to an embodiment of the present disclosure. FIG. 7B illustrates a syringe that may be used to deliver a fluid through a septum into the reservoir. FIG. 7C depicts an expanded view of the septum tip.

A porous body 702 is at a first end of the housing 704 and is in fluid communication with the reservoir 700. A septum 706 is at a second end of the housing 704 that is configured to accept a syringe 720. A syringe 720 may deliver a fluid through the septum 706 into the reservoir 700. The fluid may diffuse from the reservoir 700 through the porous body 702. The porous body 702 has a rounded (domed, convex, etc.) shape (but may be another shape such as a disk, a cup, a cylinder, a tube, a combination thereof, or the like) that extends along the axis “l”, has a thickness along the axis “t”, and a depth along the axis “d.” A three-dimensional shape along the length 1 provides more surface area for the fluid to diffuse through without significantly increasing the overall size of the device. The housing 704 includes channels 708 configured to accept one or more filaments for fixing the device to a tissue and/or to assist with delivery/removal of the device. The housing 704 is curved to substantially match an anatomy of a patient (e.g., the curve of a neck muscle, or the like) for patient comfort and to prevent device migration.

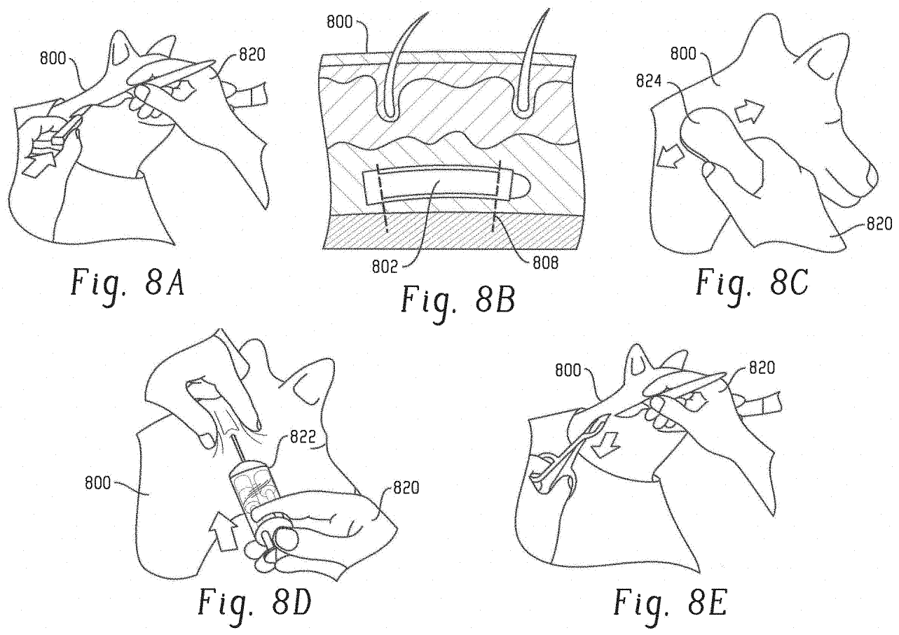

With reference to FIGS. 8A-8E, the implantable drug delivery device of FIG. 7 is illustrated as being used with a patient 800 (e.g., a domesticated dog). A medical professional 820 makes an incision in the patient 800 to implant the device 802, as shown in FIG. 8A. The device 802 is implanted into the muscle via sutures 808 within the subcutaneous tissue layer in FIG. 8B. The medical professional 820 may scan the implantation site on the patient 800 to ensure proper implantation of the device 802 and/or to locate the device 802 in FIG. 8C. The medical professional 820 loads a reservoir of the device 802 with a fluid via a syringe 822 percutaneously through the skin of the patient 800 in FIG. 8D. After use, the medical professional 820 makes an incision in the patient 800 to remove and/or replace the device 802 in FIG. 8E.

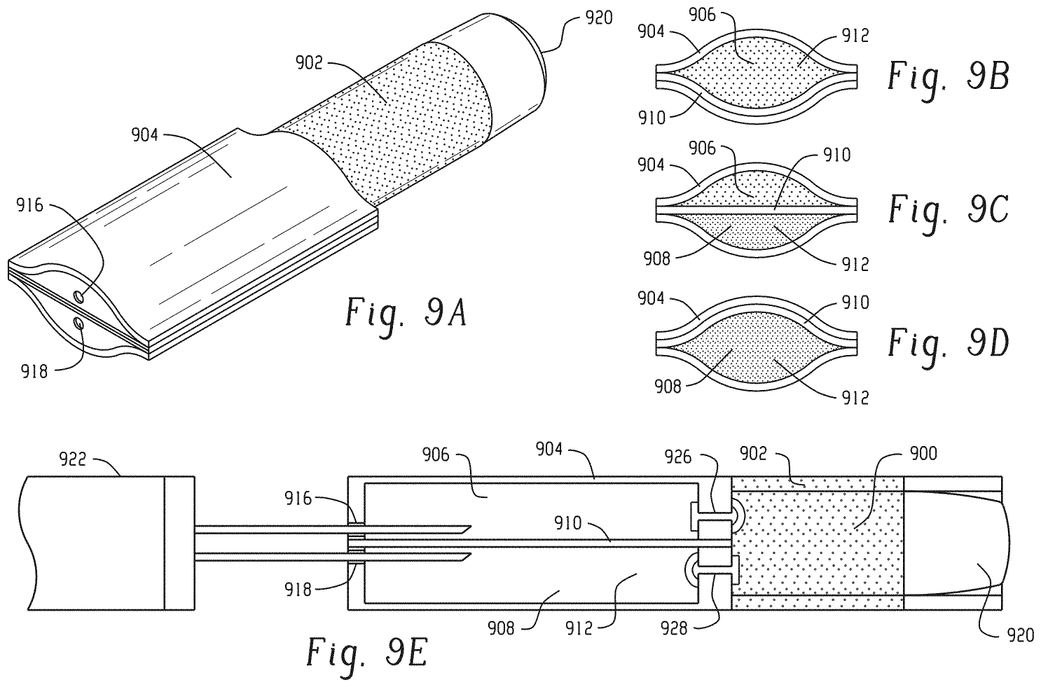

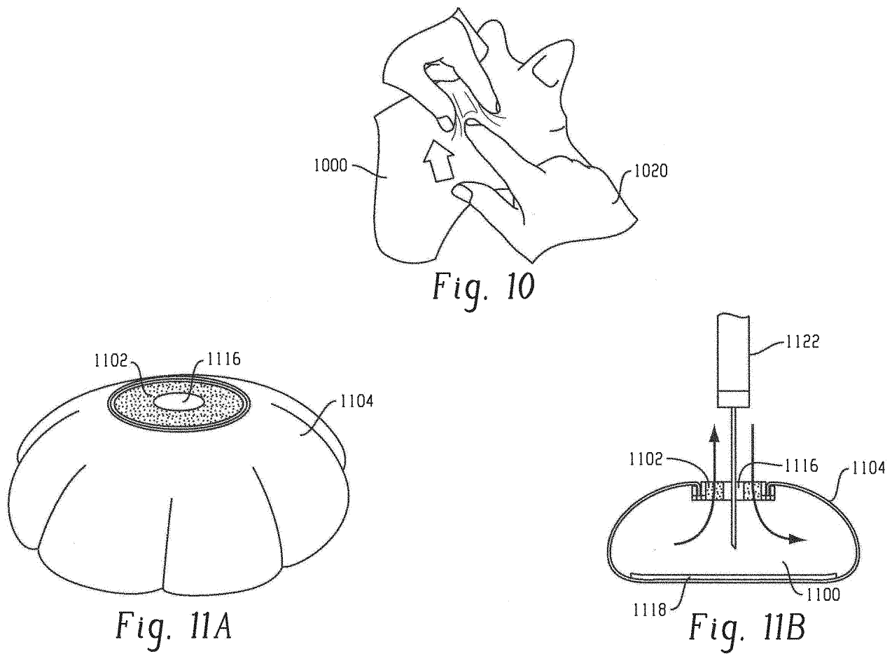

With reference to FIGS. 9A-9E, an implantable drug delivery device according to an embodiment of the present disclosure is illustrated that includes a housing 904 having a storage reservoir 912. FIG. 9A illustrates an implantable drug delivery device including a storage reservoir, according to an embodiment of the present disclosure. FIG. 9B shows the storage reservoir 912 filled with a fluid drug in fluid compartment 906. FIG. 9C shows the storage reservoir 912 partially filled with a fluid drug in fluid compartment 906 and partially filled with was in waste compartment 908. FIG. 9D shows the waste compartment 908 expanded to occupy the entire volume of the fluid reservoir 912. FIG. 9E depicts a first septum 916 is at a first end of the housing 904 and is in fluid communication with the fluid compartment 906.

In FIGS. 9A-9C, the storage reservoir 912 includes a flexible membrane 910 separating a fluid compartment 906 configured to contain a fluid drug for delivery into a patient from a waste compartment 908 configured to contain a waste. The flexible membrane 910 is flexible such that, e.g., the fluid compartment 906 may be substantially filled with a drug (e.g., as illustrated in FIG. 9B) as the flexible membrane 910 flexes so as to enlarge the fluid compartment 906 and minimize the waste compartment 908. Similarly, as the drug egresses from the fluid compartment 906, the flexible membrane 910 may minimize the fluid compartment 906 and enlarge the waste compartment 908 (e.g., as the storage reservoir 912 transitions from containing more fluid drug in FIG. 9B, to containing less fluid drug in FIG. 9C, to containing substantially no fluid drug 908 in FIG. 9D).

As fluid drug 908 egresses from the fluid compartment 906, pressure in the fluid compartment 906 is reduced (i.e., a lower volume of fluid in the same volume of space), causing the flexible membrane 910 to flex and move, reducing the volume of the fluid compartment 906. As the flexible membrane 910 reduces the volume of the fluid compartment 906, the flexible membrane 910 also increases the volume of the waste compartment 908. As the volume of the waste compartment 908 is enlarged, the pressure of the waste compartment 908 decreases, drawing in fluid (e.g., a waste fluid) into the waste compartment 908. FIG. 9E depicts a first septum 916 is at a first end of the housing 904 and is in fluid communication with the fluid compartment 906. A second septum 918 is also at the first end of the housing 904 and is in fluid communication with the waste compartment 908.

These septa 916, 918 may be used as inlets/outlets for fluids in the storage reservoir. For example, a dual syringe 922 may be inserted into the septa 916, 918 and a fluid drug may be injected into the fluid compartment 906. As the fluid drug enters the fluid compartment 906, the flexible membrane 910 flexes and moves, enlarging the fluid compartment 906 and decreasing the waste compartment 908. As the volume of the waste compartment 908 is decreased, fluid egresses into (and may possibly be drawn into) the dual syringe 922. A porous body 902 is at a second end of the housing 904.

There is a fluid reservoir 900 within the porous body 902 that is in fluid communication with the storage reservoir 912. An outlet fluid check valve 926 is in fluid communication with the fluid compartment 906 and the fluid reservoir 900 that is configured to allow flow substantially in a direction from the fluid compartment 906 to the fluid reservoir 900. There is also an inlet waste check valve 928 in fluid communication with the waste compartment 908 and the fluid reservoir 900 that is configured to allow flow substantially in a direction from the fluid reservoir 900 to the waste compartment 908. A fluid (e.g., a fluid drug) may flow passively from the fluid compartment 906, through the fluid check valve 926, into the fluid reservoir 900, and diffuse through the porous body 902. A waste (e.g., body fluid) may passively diffuse from the body of a patient through the porous body 902, into the fluid reservoir 900, through the waste check valve 928, and into the waste compartment 908.

To actively facilitate these described flow paths, a slidable member 920 at the end of the porous body 902, and substantially opposing the storage reservoir 912, is slidable within the fluid reservoir 900. The slidable member 920 is in the resting position in FIGS. 9A and 9E where the slidable member 920 is substantially external to the fluid reservoir 900. A user 1020 (FIG. 10) may engage the slidable member 920 of a device within the patient 1000 to decrease the volume of the fluid reservoir 900 by, e.g., pressing the slidable member 920 into the fluid reservoir 900. The slidable member 920 may return to the resting position after it is engaged, enlarging the volume fluid reservoir 900.

The slidable member 920 may return to the resting position by restorative forces. These restorative forces may originate from an internal spring, a restoration of a deformable material that makes up the slidable member, and/or a flow force of a fluid through an outlet check valve. After a period of time has elapsed when a substantial amount of the fluid drug has diffused from the fluid reservoir 900, through the porous body 902, and into the patient 1000, a user 1020 may engage the slidable member 920 to reload the device with a new dose of the drug. As the drug diffuses from the fluid reservoir 900 through the porous body 902, waste fluid diffuses through the porous body 902 into the fluid reservoir 900.

After the period of time, the fluid reservoir 900 is substantially filled with waste fluid. Engaging the slidable member 920 and sliding it into the fluid reservoir 900 forces the waste fluid from the fluid reservoir 900 through the waste check valve 928, and into the waste compartment 908. As the waste compartment 908 receives more waste fluid, its volume increases, pressing against the flexible member 910 to increase the volume of the waste compartment 908 and apply pressure onto the fluid compartment 906. As this pressure decreases the volume of the fluid compartment 906 on the return stroke of the slidable member 920 to the relaxed position substantially out of the fluid reservoir 900, the fluid flows from the fluid compartment 906, through the fluid check valve 926, and into the fluid reservoir 900.

The slidable member 920 may recharge the fluid reservoir 900 without the need for an injection and without the assistance of a medical professional. In another embodiment, the slidable member may instead be a compressible member that is fixed in relation to porous body. The fluid reservoir may extend into the compressible member such that when the compressible member is compressed, the fluid reservoir volume is reduced, forcing fluid into waste compartment. During decompression of the compressible member (e.g., by releasing the compressible member) the compressible member may return to its resting state, enlarging the fluid reservoir (compared to the decreased volume of the fluid reservoir when the compressible member is compressed). This enlarging of the volume of the fluid reservoir may lower the pressure within the fluid reservoir and may draw out fluid from the fluid compartment and into the fluid reservoir.

In various embodiments, a lock may be included that is configured to prevent accidental fluid dose administration while the device is implanted within a patient. The lock may be located, e.g., on or in contact with a fluid check valve or a slidable member. The lock may be configured to block fluid communication from a fluid compartment, through a fluid check valve, and into a fluid reservoir. The lock may be configured to prevent movement of a slidable member. The lock may be engaged by a user to enable or disable the lock. The lock may be engageable by a user, e.g., by a precise, rigid movement of a switch through the epidermis of the patient. The lock may be engaged by a magnet or electrical field from outside the patient's body moving in proximity to the lock.

With reference to FIGS. 11A and 11B, an implantable drug delivery device according to embodiments of the present disclosure is illustrated that includes an expandable member 1104 having a reservoir 1100 configured to contain a fluid. A porous body 1102 is at an end of the expandable member 1104 that is in fluid communication with the reservoir 1100. The porous body 1102 may be attached to the expandable member 1104 by an adhesive, pressing, crimping, or the like, and may include a rigid ring to provide structural support. The porous body 1102 is generally the shape of an annulus. A septum 1116 is disposed on the expandable member 1104 and/or within the annulus of the porous body 1102. The septum 1116 may comprise silicone rubber or the like. A syringe 1122 may be inserted through the septum 1116 to fill and/or empty the reservoir 1100. A needle-proof membrane 1118 is disposed within the expandable member 1104 in opposition to the septum 1116 such that the syringe 1122 is unable to puncture the expandable member 1104 and/or harm the patient. As the reservoir 1100 is filled, the expandable member 1104 may enlarge to an expanded configuration, increasing the volume of the reservoir 1100. As the reservoir 1100 is emptied, the expandable member 1104 may shrink to a collapsed configuration, decreasing the volume of the reservoir 1100. Because the flexible member 1104 flexes with the addition and removal of fluid from the reservoir 1100 (i.e., inflates/deflates), a single septum 1106 may be used to either supply or remove fluid from the reservoir 1100. A substantially uncompressible housing (not shown) may be disposed about the flexible member to protect the flexible member 1104 from being engaged by out-of-patient-body forces that may traumatize the flexible member 1104, causing it to force an undesirable release of fluid through the porous body 1102. The housing may be, e.g., a cage or screen with apertures that may be small enough to prevent foreign bodies from compressing the expandable member 1104. The housing may prevent outside pressure from exerting directly onto the expandable member 1104, preventing pressurized flow of fluid from the reservoir 1100 and a possible overdose of fluid to the patient. In various embodiments, exemplary materials for an expandable member may include any biocompatible material such as a polymer, silicone, rubber, or the like. An expandable member may have, e.g., a diameter of about 1 inch (25.4 mm) to about 3 inches (76.2 mm) depending on the size of the patient.

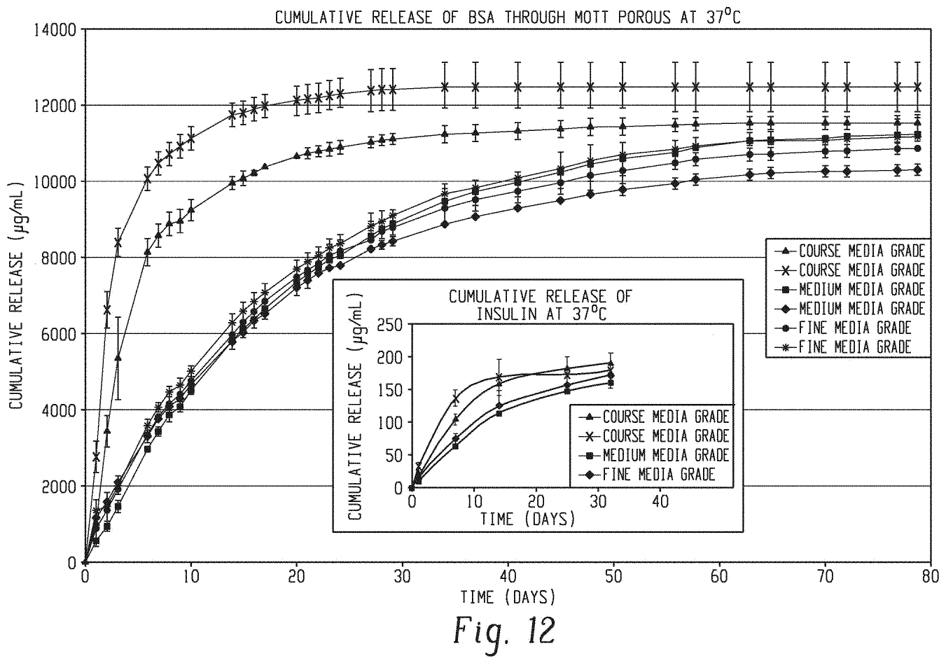

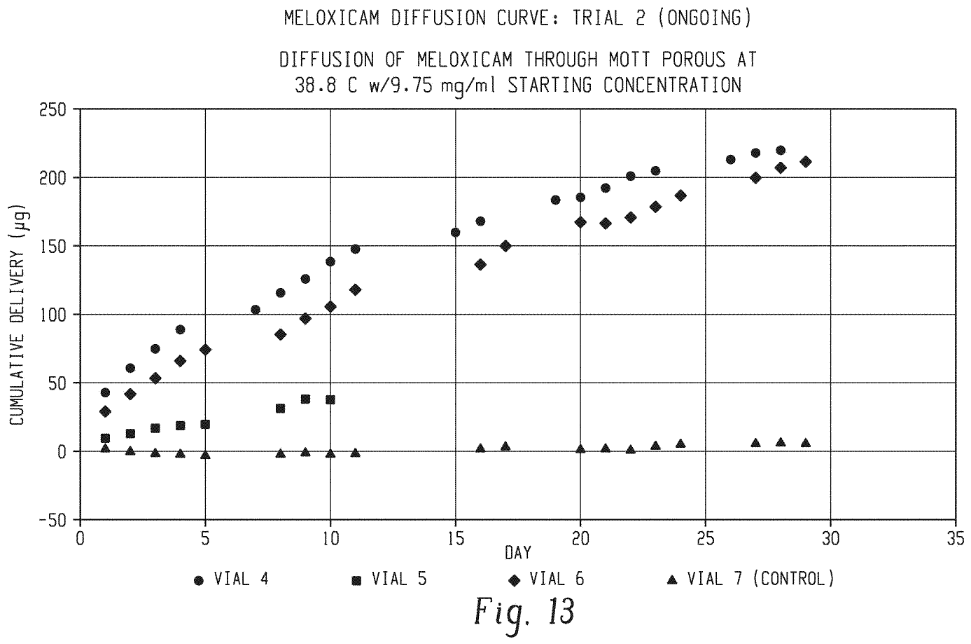

FIGS. 12 and 13 illustrate charts of various fluid delivery rates through porous bodies according to embodiments of the present disclosure. Each chart plots example fluids released (delivered, diffused, etc.) into a solution over time. The y-axis generally displays the amount of fluid released over an amount of time, which is displayed along the x-axis. Pre-clinical trials of various embodiments were designed to determine material compatibility, verify that the porous media does not foul (plug prematurely), quantify the rate of drug delivery, and verify the lifetime of the device (e.g., when a device will substantially cease release/delivery/diffusion of a fluid).

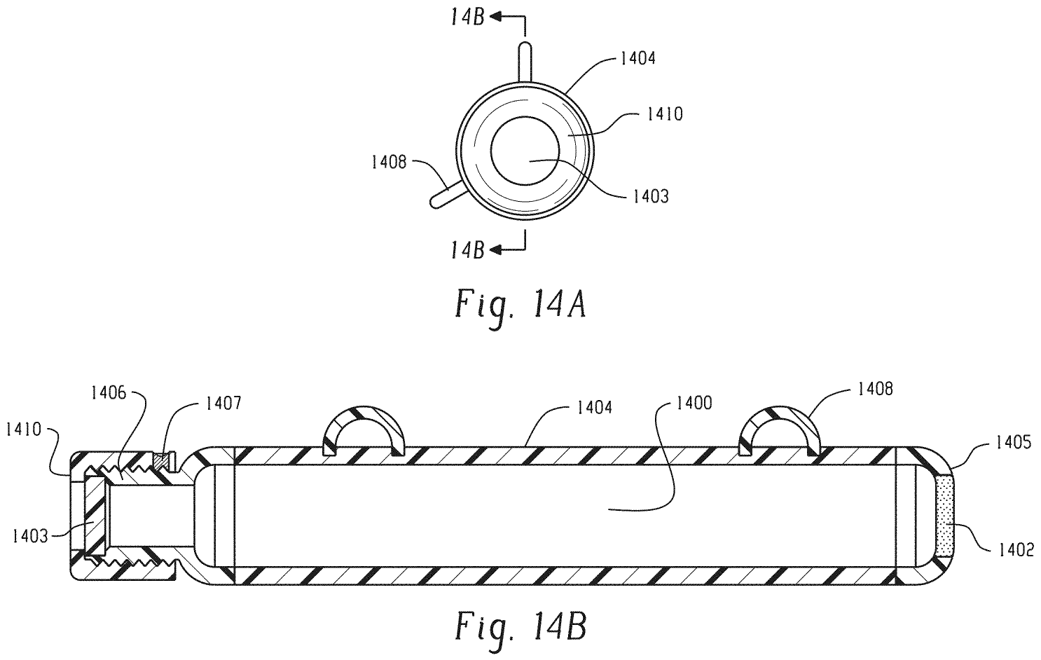

With reference to FIGS. 14A and 14B, an implantable drug delivery device according to an embodiment of the present disclosure is illustrated that includes a reservoir 1400 within a housing 1404. The housing 1404 is substantially cylindrical and contains a fluid within the reservoir 1400. The housing 1404 has a neck 1406 that is threaded and has an aperture that is in fluid communication with the reservoir 1400. The housing 1404 also has a bottom 1405 that includes an aperture in fluid communication with the reservoir 1400. A septum 1403 is compressed against the neck 1406 by a cap 1410 that is threaded onto the housing 1400 and is also secured by a set screw 1407. A porous body 1402 is within the aperture of the bottom 1405 of the housing such that there is a fluid flow path from the reservoir 1400 through the porous body 1402. The reservoir 1400 may be filled, emptied, and/or refilled through the septum 1403. In this embodiment, surgical loop filaments 1408 are attached to the housing 1404 for manipulating and/or securing the housing 1404.

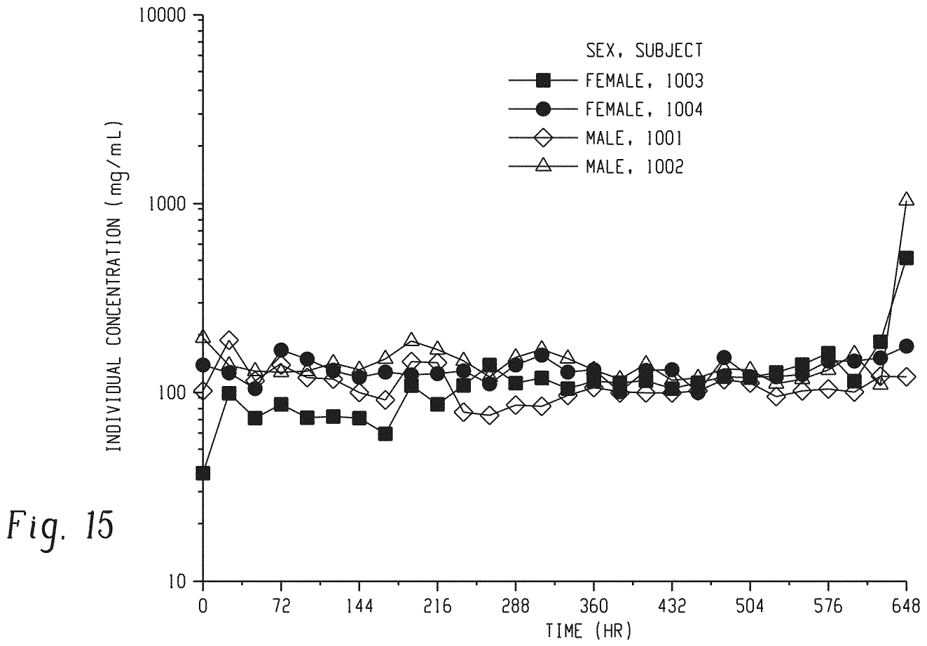

FIG. 15 is a graph that depicts the results for MELOXICAM® delivery to male and female dogs using the device detailed herein. The data clearly shows a consistent delivery rate over 612 hours (25 days) of just over 100 nanograms per milliliter (ng/ml) for that time period. The rises at the end were due to refilling of two of the devices (one female and one male dog) towards the end of the study.

In various embodiments, porous bodies may comprise a multitude of shapes and densities. A porous body may have a symmetrical or asymmetrical cross-section. A cross-section of a porous body may be substantially round, ellipsoidal, rectangular, oblong, or the like. Laser additive manufacturing technology (“LAMT”) may be used to create porous media for devices herein. As used herein, additive manufacturing refers to a 3D printing process whereby successive layers of material are formed to create an object of a desired shape. Laser additive manufacturing refers to additive manufacturing techniques that employ a laser to melt, soften, sinter or otherwise affect the material used in the object being manufactured. By varying material and manufacturing process specifications and conditions, a desired and tailored pore size, morphology, and distribution may be produced.

The resultant porous structure may be used as is, or it may be joined or otherwise fabricated with a solid full density component to complete a finished product. As used herein, “solid” and “substantially non-porous” are used synonymously to mean a component does not exhibit a through-thickness interconnected porosity. The laser additive manufacturing processes of the present invention are used to create porous structures, solid structures, and structures that have both porous and solid portions that are integrally formed together. Generally, the laser additive manufacturing processes described herein, when used in accordance with the present invention, are used to create unique porous structures that result in lower pressure drop properties for a given pore size when compared with conventional powder compacted/sintered porous structures. LAMT offers the additional ability to create finished form parts in customized materials and geometries, and to vary the pore structure within a product for customized and unique properties.

The porous media of the present invention that are produced from LAMT techniques are long lasting and provide efficient particle capture, flow restrictor-control, wicking, and fluid contacting. The LAMT processes of the present invention may use a unique, controlled powder particle recipe (spherical and/or irregular shaped powder) that serves as the feed material for the products to be manufactured. The particles can be joined through the use of laser technology to form an interconnected pore structure that provides uniformly sized predicted sintered pores. The various pores size that can be produced for specific applications can be grouped or classified in media or product grades of 0.1 to 200 micrometers, which represents an average pore sizes of a manufactured product.

Exemplary devices, systems, and methods with which embodiments of the present disclosure may be implemented include, but are not limited to, those described in U.S. Pat. No. 7,112,234, and U.S. patent application Ser. No. 15/395,528, each of which are herein incorporated by reference in their entirety. Exemplary devices described therein may be modified to incorporate embodiments or features of the present disclosure.

In an embodiment, the implantable drug delivery device is of a size and shape that permits it to be implanted under the skin (subcutaneously) of the patient without being detectable by simple human touch. In other words, the implantable drug delivery device is small enough that when a human being passes his/her hand over the patient skin at the site of implantation of the device, it cannot be felt. The device does not protrude beyond the skin and alternatively does not cause a protrusion of the skin surface even as it lies below the skin

Despite the fact that the implantable drug delivery device does not protrude from the skin or does not cause a protrusion in the skin it may be locatable by a physician using a detector. The detector may include a magnetic sensor, an electrical sensor, and so on. The ability of locate the device location facilitates refilling of the reservoir and/or removal of waste products and byproducts.

In an embodiment, the implantable drug delivery device is tiny enough to facilitate ease of surgical insertion without requiring extensive surgery on the patient. A small device may be inserted below the skin with only a small incision thus preventing the formation of large disfiguring scars which may require plastic surgery. In addition, the device may be capable of being secured to the sutures so that it does not migrate once inserted into the body of the patient. It may also be provided with identification features that make it easily locatable once inserted subcutaneously and that permit only permitted users to activate the device. In other words, the device may be provided with a code that can be known and used only by permitted users. This prevents accidental drug delivery by unauthorized users.

In an embodiment, the implantable drug delivery device may contain more than one reservoir (i.e., may contain a plurality of reservoirs) that can be used to deliver more than one type of drug simultaneously or sequentially. The one or more type of drugs may include recuperative drugs, restorative drugs, antidotes, or the like. At least one of the reservoirs can contain an antidote to minimize the effect of allergic reactions. This is detailed below. The plurality of reservoirs may be sized to deliver a synergistic volumetric combination of drugs for efficacious recovery. In another embodiment, each reservoir may be independently controlled by a microprocessor (not shown) that can deliver a different dosage of each drug to the patient. These dosages may be varied independently of each other during each instance of drug delivery. For example, during a first delivery, a first drug (contained in a first reservoir) may be delivered at twice the rate of a second drug (contained in a second reservoir). During a second delivery, the first drug may be delivered at three times the rate of the second drug, and so on.

In yet another embodiment, the drug dosage from the implantable drug delivery device may delivered at varying rates depending upon the body mass index (BMI) of the patient. The microprocessor can control as well as communicate with the device remotely. The microprocessor can communicate with the device via microwaves and/or radiowaves, and the like. In an embodiment, the microprocessor can control the device and can communicate with the device using WiFi, Bluetooth, or the like, or a combination thereof. The device may itself be programmable or alternatively, may be programmed via a microprocessor.

In an embodiment, the implantable drug delivery device may be provided with a quick stop mechanism that is operative to immediately stop drug delivery from the device to the patient if an adverse reaction to the drug is observed to occur. The quick stop mechanism may be operated manually or electronically via a microprocessor. In an embodiment, the quick stop mechanism may include a valve that is in communication with a microprocessor via radio waves, microwaves, and the like. The microprocessor can issue a command to the quick stop mechanism that will activate the valve and stop the drug delivery.

In yet another embodiment, the implantable drug delivery device may contain an additional reservoir (not shown) that may contain an antidote that can be delivered to the patient to quickly stop and/or reverse any adverse effects from the drug delivery. The antidote delivery may also be activated manually or electronically via the microprocessor. The microprocessor can issue a command to an activation mechanism that will activate delivery of the antidote to the patient.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. An implantable drug delivery device comprising:
a housing;
a reservoir within the housing configured to contain a fluid; and
a homogenous porous body having non-uniform pores of about 0.1 microns to about 100 microns at a first end of the housing in fluid communication with the reservoir.

2. The implantable drug delivery device of claim 1, further comprising a septum at a second end of the housing in fluid communication with the reservoir.

3. The implantable drug delivery device of claim 1, wherein the reservoir extends into the porous body.

4. The implantable drug delivery device of claim 1, wherein the porous body comprises a convex body.

5. The implantable drug delivery device of claim 1, further comprising one or more filaments disposed on the drug delivery device configured to attach the drug delivery device to a tissue.

6. The implantable drug delivery device of claim 1, wherein the housing is curved to substantially match an anatomy of a patient.

7. The implantable drug delivery device of claim 1, further comprising a channel disposed on the housing configured to accept a suture configured to anchor the implantable drug delivery device to a tissue.

8. The implantable drug delivery device of claim 1, wherein the porous body comprises a material selected from the group consisting of stainless steel, glass, titanium, a biocompatible metal alloy, ceramic, and polymers.

9. The implantable drug delivery device of claim 1, wherein the porous body comprises a selective laser sintered metal.

10. The implantable drug delivery device of claim 1, wherein the porous body comprises an additive metal.

11. The implantable drug delivery device of claim 1, wherein the porous body makes up the housing.

12. The implantable drug delivery device of claim 1, wherein the porous body is configured such that the fluid diffuses from the reservoir through the porous body at a constant mass amount per an amount of time over a an extended period of time.

13. The implantable drug delivery device of claim 12, wherein the period of time is about 90 days.

14. The implantable drug delivery device of claim 1, wherein the device is configured to be of a shape and size that does not permit its detection by human touch when located subcutaneously in a patient.

15. The implantable drug delivery device of claim 1, wherein the device does not protrude from the skin of the patient when located subcutaneously in a patient.

16. The implantable drug delivery device of claim 1, wherein the device does cause a protrusion to be felt on a skin of the patient when located subcutaneously in a patient.

17. The implantable drug delivery device of claim 1, wherein the device is locatable under the skin using a detector.

18. The implantable drug delivery device of claim 1, wherein the device can be replenished with a drug and/or have products and byproducts evacuated from it.

19. The implantable drug delivery device of claim 1, where the device further comprises one or more additional reservoirs that contain additional fluid and wherein these additional reservoirs are operative to deliver a recuperative drug to the patient.

20. The implantable drug delivery device of claim 1, where at least one of the additional reservoirs are operative to deliver an antidote to the patient.

21. The implantable drug delivery device of claim 1, where the device is controlled remotely.

22. An implantable drug delivery device comprising:
a housing;
a storage reservoir within the housing;
a flexible membrane within the storage reservoir separating a fluid compartment configured to contain a fluid for delivery and a waste compartment configured to contain a waste;
a first septum at a first end of the housing in fluid communication with the fluid compartment;
a second septum at the first end of the housing in fluid communication with the waste compartment;
a porous body at a second end of the housing; and
a fluid reservoir within the porous body in fluid communication with the storage reservoir.

23. The implantable drug delivery device of claim 22, further comprising
a fluid check valve in fluid communication with the fluid compartment and the fluid reservoir, the fluid check valve configured to allow flow substantially in a direction from the fluid compartment to the fluid reservoir; and
a waste check valve in fluid communication with the waste compartment and the fluid reservoir, the waste check valve configured to allow flow substantially in a direction from the fluid reservoir to the waste compartment.

24. The implantable drug delivery device of claim 23, further comprising a lock configured to block fluid communication from the fluid compartment, through the fluid check valve, and into the fluid reservoir.

25. The implantable drug delivery device of claim 22, further comprising:
a slidable member at an end of the porous body substantially opposing the storage reservoir and slidable within the fluid reservoir;
wherein the slidable member is configured to be user-engageable to decrease a volume of the fluid reservoir;
wherein the slidable member has a resting configuration where the slidable member is substantially external to the fluid reservoir; and
wherein the slidable member has an engaged configuration where the slidable member is substantially within the fluid reservoir.

26. The implantable drug delivery device of claim 25, further comprising a lock configured to prevent the slidable member from sliding within the fluid reservoir.

27. An implantable drug delivery device comprising:
an expandable member;
a reservoir within the expandable member configured to contain a fluid;
a porous body at a first end of the expandable member in fluid communication with the reservoir;
wherein the expandable member has an expanded configuration when a fluid is delivered into the reservoir; and
wherein the expandable member has a collapsed configuration when a fluid is removed from the reservoir.

28. The implantable drug delivery device of claim 27, further comprising a septum disposed on the expandable member.

29. The implantable drug delivery device of claim 28, further comprising a puncture-proof membrane disposed within the expandable member substantially opposing the septum.

30. The implantable drug delivery device of claim 28, wherein the porous body is an annulus about the septum.

31. The implantable drug delivery device of claim 27, further comprising a rigid housing about the expandable member.
